(19) United States
(12) Patent Application Publication
(10) Pub. No.: US 2020/0345759 A1
(43) Pub. Date: Nov. 5, 2020

(54) METHODS OF TREATING GALECTIN-3 DEPENDENT DISORDERS

(71) Applicants: Pieter Muntendam, (US); RENSSELAER POLYTECHNIC INSTITUTE, Troy, NY (US)

(72) Inventors: Robert Linhardt, Albany, NY (US); Jing Zhao, Troy, NY (US); Fuming Zhang, Watervliet, NY (US); Pieter Muntendam, Boxford, MA (US)

(21) Appl. No.: 16/933,043

(22) Filed: Jul. 20, 2020

Related U.S. Application Data
(63) Continuation of application No. PCT/US2019/014176, filed on Jan. 18, 2019.
(60) Provisional application No. 62/619,857, filed on Jan. 21, 2018.
(60) Provisional application No. 62/792,931, filed on Jan. 16, 2019.

(51) Int. Cl. A61K 31/715; A61K 36/42

(52) U.S. Cl. CPC A61K 31/715; A61K 36/42

(57) ABSTRACT

A therapeutic composition includes a polysaccharide, isolated from a member of the genus Cucurbita, e.g., pumpkin, having a backbone including alternating α-L-rhamnosyl (α-L-Rhap) and α-D-galactopyranosyluronic acid (α-D-GapA) residues, and a side chain attached to the backbone including β-D-galactan (β-D-Galp), α-L-arabinofuranosyl (α-L-Araf), or combinations thereof, and a pharmaceutically acceptable excipient. A β-D-Galp side chain is attached to the backbone at the C-4 carbon of at least one α-L-Rhap of the backbone. At least one α-L-Araf is attached to the β-D-Galp side chain. The α-L-Araf is attached to the β-D-Galp side chain via the C-3 carbon of the β-D-Galp. The polysaccharide is effective for treating a galectin-3 dependent disorder by binding to the carbohydrate recognition domain of galectin-3, resulting in inhibition of galectin-3 activity.

### CROSS REFERENCE TO RELATED APPLICATION(S)

This application claims the benefit of U.S. Provisional Application Nos. 62/619,857, filed Jan. 21, 2018, and 62/792,931, filed Jan. 16, 2019, which are incorporated by reference as if disclosed herein in their entireties.

### STATEMENT REGARDING FEDERALLY SPONSORED RESEARCH OR DEVELOPMENT

This invention was made with government support under grant nos. 6910301-17-0009 awarded by the National Institutes of Health. The government has certain rights in the invention.

### BACKGROUND

Galectin-3 (also referred to herein as gal-3, formerly known as the Mac-2 antigen) is a protein belonging to a specific sub-family of carbohydrate binding proteins (lectins) that recognize β-galactosides. Galectin-3 is the only family member that is composed of a glycine/proline rich N-terminal repeated sequence and a C-terminal carbohydrate-binding domain. Galectin-3 is a pleiotropic lectin that plays an important role in cell proliferation, adhesion, differentiation, angiogenesis, and apoptosis. Galectins possess a carbohydrate recognition domain (CRD). The CRDs of various galectins differ in amino acid sequence outside of the conserved residues mediating specificity to different glycan ligands between galectins. Galectin-3 has both intracellular functions and extracellular functions and is actively secreted via a non-canonical pathway into the extracellular space and into circulation. Binding of carbohydrates to the CRD can result in modulation of galectin-3 activity in-vitro and in-vivo.

Galectin-3 has key roles in fibrogenesis affecting various organ systems including renal, pulmonary and cardiovascular systems. Fibrosis plays a key role in diseases such as heart failure, chronic kidney disease, chronic lung disease, and chronic vascular disease including abdominal arterial aneurysm and vascular stiffening. Galectin-3 is expressed in a variety of cell types as an immune response to microbial invasion that may also include inflammation as a response affecting the brain, eye, skin, joints and other organs of the body. Studies have also revealed that galectin-3 has a role in cancer. Currently, there are no approved therapeutic agents targeting galectin-3 for the prevention or treatment of diseases and disorders that include cardiovascular and renal diseases affecting a large part of the population. There exists a serious gap in the therapeutic strategy against galectin-3-mediated diseases and disorders. Hence, there is a need for a promising therapeutic strategy against galectin-3-mediated diseases and disorders.

### SUMMARY

Some embodiments of the present disclosure are directed to a therapeutic composition including a polysaccharide having a backbone including alternating α-L-rhamnosyl (α-L-Rhap) and α-D-galactopyranosyluronic acid (α-D-GalpA) residues, and a side chain attached to the backbone including β-D-galactan (β-D-Galp), α-L-arabinofuranosyl (α-L-Araf), or combinations thereof, and a pharmaceutically acceptable excipient. In some embodiments, a β-D-Galp side chain is attached to the backbone at the C-4 carbon of at least one α-L-Rhap of the backbone. In some embodiments, at least one α-L-Araf is attached to the β-D-Galp side chain. In some embodiments, the α-L-Araf is attached to the β-D-Galp side chain via the C-3 carbon of the β-D-Galp. In some embodiments, the polysaccharide isolated from a member of the genus Cucurbita.

Some embodiments of the present disclosure are directed to a method of treating a galectin-3 dependent disorder including determining that a patient has a galectin-3 dependent disorder and administering to the patient a therapeutically effective dose of the therapeutic composition. In some embodiments, the galectin-3 dependent disorder includes galectin-3-mediated diseases and disorders including fibrosis, inflammation, organ damage, impaired organ function, cardiovascular disease, kidney disease, lung disease, cancers, heart disease, elevated blood galectin-3 level, elevated levels of the one or more collagen turnover markers, or combinations thereof. Some embodiments of the present disclosure include a method of isolating a polysaccharide including suspending an amount of plant material in an alkali hydroxide solution, heating the suspension, and isolating a polysaccharide-including supernatant layer from the suspension.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic drawing of a polysaccharide according to some embodiments of the present disclosure;

FIG. 2 is a chart of a method for isolating a polysaccharide according to some embodiments of the present disclosure;

FIG. 3 is a chart of a method for treating a galectin-3 dependent disorder according to some embodiments of the present disclosure;

FIG. 4 portrays a monosaccharide composition determined by reversed-phase high-performance liquid chromatography for a polysaccharide according to some embodiments of the present disclosure;

FIG. 5 portrays an ¹H NMR spectrum for a polysaccharide according to some embodiments of the present disclosure;

FIG. 6 portrays an ¹³C NMR spectrum for a polysaccharide according to some embodiments of the present disclosure;

FIG. 7 portrays a heteronuclear single quantum correlation for a polysaccharide according to some embodiments of the present disclosure;

FIG. 8 portrays a ¹H-¹H correlation (COSY) spectrum for a polysaccharide according to some embodiments of the present disclosure;

FIG. 9 portrays a heteronuclear multiple bond correlation (HMBC) for a polysaccharide according to some embodiments of the present disclosure;

FIG. 10 portrays a surface plasmon resonance (SPR) sensorgram of pectic polysaccharide and Ricnus Communis Agglutinin I (RCA₁₂₀) binding showing a smooth binding curve between pectin polysaccharide and RCA₁₂₀; and

FIG. 11 portrays an SPR sensorgram of pectic polysaccharide and RCA₁₂₀ binding with pectic polysaccharide concentrations.

### DETAILED DESCRIPTION

Some aspects of the disclosed subject matter include a therapeutic composition including a polysaccharide effective to bind to and inhibit the activity of a galectin-3 protein. In some embodiments, the polysaccharide is a compound including a plurality of long chains of sugar units linked together by glycosidic linkages, which after breakdown or hydrolysis yields one or more fragments that bind to the galectin-3 carbohydrate recognition domain resulting in inhibition of galectin-3 activity. As used herein, a “compound” refers to the compound itself and its pharmaceutically acceptable salts, hydrates and esters, unless otherwise understood from the context of the description or expressly limited to one particular form of the compound, i.e., the compound itself, or a pharmaceutically acceptable salt, hydrate or ester thereof. In some embodiments, the polysaccharide in the therapeutic composition is in long chain form to be broken-down/hydrolyzed subsequent to administration to a patient. In some embodiments, the polysaccharide in the therapeutic composition is broken-down/hydrolyzed prior to administration to the patient. In some embodiments, the therapeutic composition also includes pharmaceutically acceptable adjuvants, diluents, excipients, carriers, or combinations thereof. In some embodiments, the therapeutic composition includes one or more additional active ingredients, e.g., angiotensin-converting enzyme (ACE) inhibitors, antiplatelet agents, angiotensin II receptor blockers, beta blockers, calcium channel blockers, diuretics, vasodilators, digitalis preparations, statins, or combinations thereof. In some embodiments, the therapeutic composition is configured for administration enterally or parenterally, e.g., oral, sublingual, rectal, intavenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, etc., or combinations thereof.

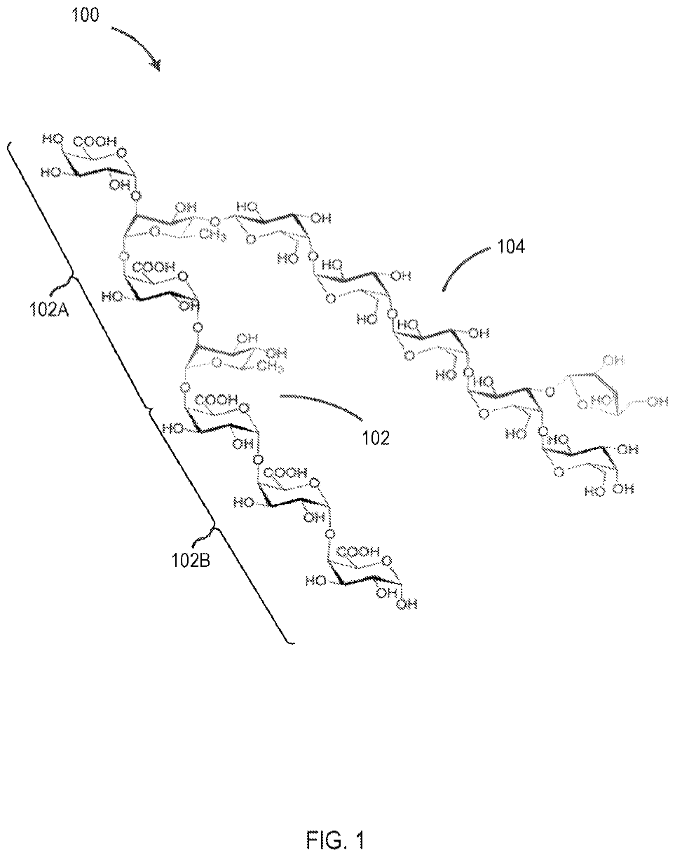

Referring now to FIG. 1, in some embodiments of the present disclosure, the polysaccharide 100 has a backbone 102 including an rhamnogalacturonan I (RG-I) domain 102A. In some embodiments, the backbone 102 includes RG-I domain 102A and a homogalacturonan (HG) domain 102B. In some embodiments, RG-I domain 102A includes α-L-rhamnosyl (α-L-Rhap) and α-D-galactopyranosyluronic acid (α-D-GalpA) residues. In some embodiments. RG-I domain 102A includes alternating α-L-Rhap and α-D-GalpA residues. In some embodiments, RG-I domain 102A includes alternating blocks of α-L-Rhap and α-D-GalpA residues. In some embodiments, the HG domain is comprised substantially of 1,4-α-D-GalpA residues. In some embodiments, the HG domain includes one or more functional group substitutions.

In some embodiments, a side chain 104 is attached to the backbone. In some embodiments, a plurality of side chains 104 are attached to the backbone. In some embodiments, side chains 104 are attached to RG-I domain 102A of the backbone 102. In some embodiments, side chains 104 include β-D-galactan (β-D-Galp), α-L-arabinofuranosyl (α-L-Araf), or combinations thereof. In some embodiments, side chain 104 is attached to the backbone 102 at the C-4 carbon of at least one α-L-Rhap residue. In some embodiments, the side chain is a β-D-Galp side chain. In some embodiments, one or more α-L-Araf residues are attached to the β-D-Galp side chain. In some embodiments, the one or more α-L-Araf residues are attached to the β-D-Galp side chain via the C-3 carbon of the β-D-Galp. In some embodiments, the polysaccharide has a structure according to the following Formula I:

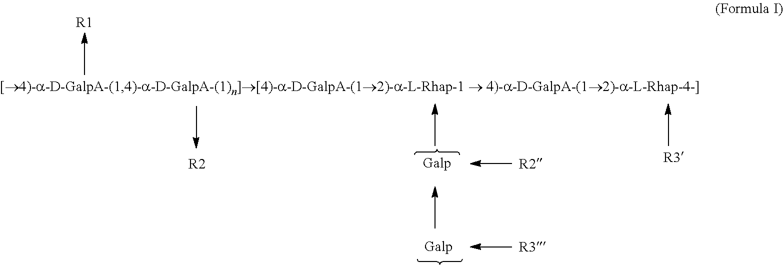

wherein R1 is an H or O-alkyl group, R2 is an H or O-acetyl group, and R3′, R3″, and R3″′ are H, α-L-Araf, or combinations thereof.

In some embodiments, the molecular weight of polysaccharide 100 is about 5 kDa to about 70 kDa. In some embodiments, the molecular weight of polysaccharide 100 is about 20 kDa to about 30 kDa. In some embodiments, the molecular weight of polysaccharide 100 is about 20 kDa to about 25 kDa. In some embodiments, the molecular weight of polysaccharide 100 is about 5 kDa to about 25 kDa. In some embodiments, the molecular weight of polysaccharide 100 is about 17 kDa to about 23 kDa. In some embodiments, the molecular weight of polysaccharide 100 is 17.5 kDa.

In some embodiments, polysaccharide 100 is isolated from a plant material, as will be discussed in greater detail below. In some embodiments, the plant material is a member of the genus Cucurbita. In some embodiments, polysaccharide 100 is isolated from C. moschata, C. argyrosperma, C. ficifolia, C. maxima, and C. pepo. In some embodiments, polysaccharide 100 is produced by a chemical processing method, enzymatic processing method, physical processing method, chemical synthesis, recombinant DNA technology, or combinations thereof. In some embodiments, the recombinant DNA technology involves fungi, bacteria, algae, another suitable host, or combinations thereof.

In some embodiments, polysaccharide 100 has a galectin-3 binding affinity greater than that of potato galactan. In some embodiments, polysaccharide 100 inhibits galectin-3 activity at concentrations of the polysaccharide below 2 μM. In some embodiments, polysaccharide 100 inhibits galectin-3 activity at concentrations of the polysaccharide at about 1.26 μM. In some embodiments, polysaccharide 100 is given one or more modifications concurrent with or subsequent to isolation from the plant material. In some embodiments, the one or more modifications include alkylation, amidation, quaternization, thiolation, sulfation, oxidation, chain elongation, e.g., cross-linking, grafting, etc., depolymerization by chemical, physical, or biological processes including enzymatic process, etc., or combinations thereof.

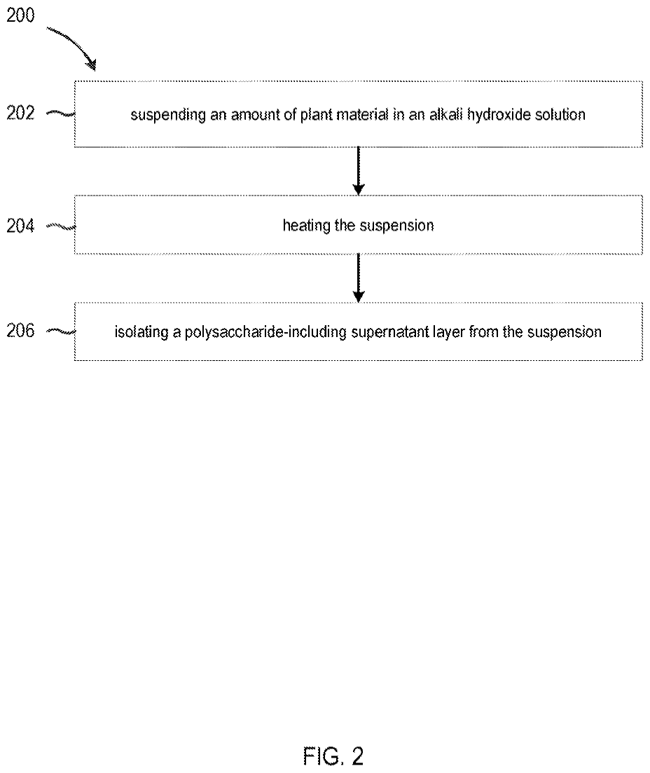

Referring now to FIG. 2, some aspects of the disclosed subject matter include a method 200 of isolating a polysaccharide. At 202, an amount of plant material is suspended in an alkali hydroxide solution. In some embodiments, the alkali hydroxide is NaOH, KOH, or combinations thereof. At 204, the suspension is heated, e.g., to about 50° C. At 206, a polysaccharide-including supernatant layer is isolated from the suspension, e.g., via centrifugation.

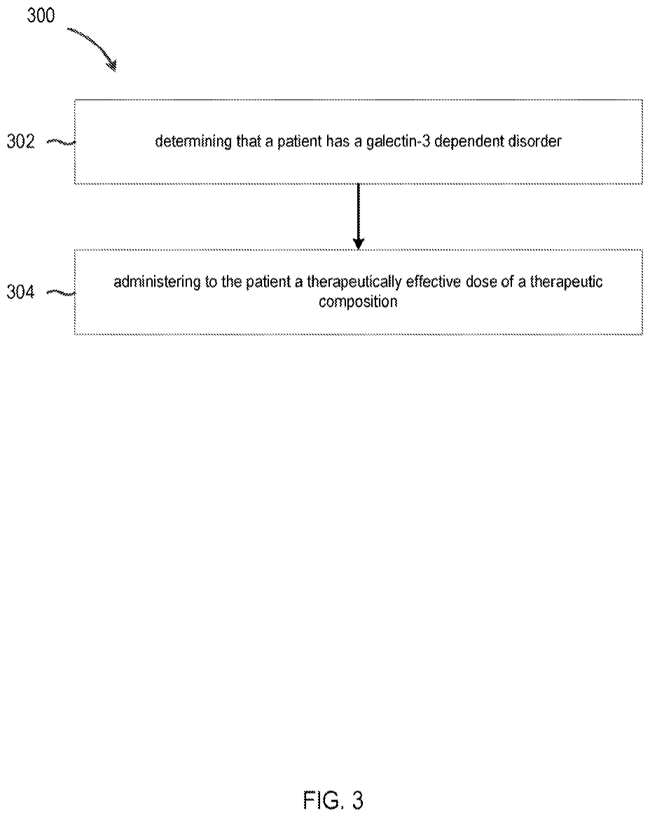

Referring now to FIG. 3, some aspects of the disclosed subject matter include a method 300 of treating a galectin-3 dependent disorder in a patient. At 302, it is determined that the patient has a galectin-3 dependent disorder. At 304, a therapeutically effective dose of the therapeutic composition is administered to the patient. In some embodiments, the therapeutically effective dose includes sufficient polysaccharide to inhibit galectin-3 activity. By inhibiting galectin-3 activity, the polysaccharide inhibits and can thus prevent, arrest, reduce, and/or treat galectin-3 dependent disorders. When administered for the treatment or inhibition of a particular galectin-3 dependent disorder, it is understood that an effective dosage can vary depending upon many factors such as the particular compound or therapeutic composition utilized, the mode of administration, and severity of the condition being treated, various physical factors related to the individual being treated, etc. In therapeutic applications, a compound or therapeutic composition of the present disclosure can be provided to a patient already suffering from a disease, for example, heart failure, in an amount sufficient to at least partially ameliorate the symptoms of the disease and its complications and halt or slow down the disease's progression. If administered to a human suffering from the condition prior to clinical manifestation, the administration of a therapeutic composition may prevent the first clinical manifestation or delay its onset.

In some embodiments, the galectin-3 dependent disorder includes galectin-3-mediated diseases and disorders including fibrosis, inflammation, organ damage, impaired organ function, cardiovascular disease, kidney disease, lung disease, cancers, heart disease, elevated blood galectin-3 level, elevated levels of the one or more collagen turnover markers, or combinations thereof. In some embodiments, the galectin-3-mediated diseases and disorders include heart failure; chronic kidney disease; chronic lung disease; chronic vascular disease, e.g., abdominal arterial aneurysm and/or vascular stiffening; neurological or neurodegenerative disease or conditions, e.g., Alzheimer's disease, Amyotrophic Lateral Sclerosis (ALS), Parkinson's disease, or Multiple Sclerosis; ischemia; reperfusion; hypoxia; atherosclerosis; ureteral obstruction; diabetes; complications of diabetes; arthritis; liver damage; insulin resistance; diabetic nephropathy; acute renal injury; chronic renal injury; acute or chronic renal injury due to exposure to radio contrast dyes or any such agents; metabolic syndromes; an ophthalmic disease or condition, e.g., dry eye, diabetic retinopathy, cataracts, retinitis pigmentosa, glaucoma, macular degeneration, choroidal neovascularization, retinal degeneration, oxygen-induced retinopathy, cardiomyopathy, ischemic heart disease, heart failure, hypertensive cardiomyopathy, vessel occlusion, vessel occlusion injury, myocardial infarction, coronary artery disease, or oxidative damage. In some embodiments, the collagen-turn-over marker includes at least one of Collagen type 1 C-terminal propeptide (CICP), Collagen type 1 C-terminal telopeptide (ICTP), Collagen type 1 N-terminal propeptide (PINP), and Collagen type III N-terminal propeptide (PIIINP), or a dependent or related marker. The disorder may also be present in an early or subclinical form. In an embodiment, the method includes reducing one or more collagen-turn-over markers.

### EXAMPLES

Preparation of the Polysaccharide Composition

Pumpkin residue (50 g) was suspended in 1 M NaOH solution as extracting agent with a solid-liquid ratio of 1/30 (w/v). The mixture was warmed at 50° C. for 4 hours with stirring. The supernatant layer was obtained by centrifugation at 8000×g for 15 min, neutralized, and then concentrated to 200-300 mL by evaporation. The protein in the extract was removed by Sevag reagent. Ethanol was then added to the solution to obtain a final concentration of 80 vol % and the polysaccharide was precipitated at 4° C. for 12 h. The polysaccharide precipitate was recovered by centrifugation at 8000×g for 25 min, dialyzed (membrane cut-off of 1000 Da) and lyophilized. The precipitated polysaccharide was applied to a Diethylaminoethanol (DEAE) Sepharose Fast Flow gel column (2.5×8 cm) and eluted by three column volumes of 0, 0.1, 0.2, 0.3, 0.5 M NaCl. Each fraction was collected and precipitated with ethanol, dialyzed (membrane cut-off of 1000 Da) and then lyophilized.

Size exclusion Chromatography (SEC) using TSK-GEL G4000SWXL (30 cm×7.8 mm) and G3000SWXL (30 cm×7.8 mm) in series and a refractive index (RI) detector was utilized for evaluation of the homogeneity and average molecular weight (Mw) of pumpkin pectic polysaccharide. The mobile phase was 7.71 g ammonia acetate and 0.2 g sodium azide in 1 L water and a flow rate of 0.6 mL/min (400 C) was used in SEC. Pumpkin pectic polysaccharide was dissolved in mobile phase (5 mg/mL) and filtered through a 0.45 μm membrane filter. A series of molecular weight standards (1, 5, 10, 25, and 50 kDa) were used. Sample (20 μL) was injected and the data obtained were analyzed by HPLC using LC solution Software (Shimadzu Scientific Instruments, MA, USA). The yields of the crude polysaccharide and the 0.2 M NaCl fraction were 5 g and 1.25 g from 100 g of pumpkin residue. The purity was evidenced by the elution of a single symmetrical peak in SEC at ˜25 min.

Monosaccharide Composition of Isolated Polysaccharide

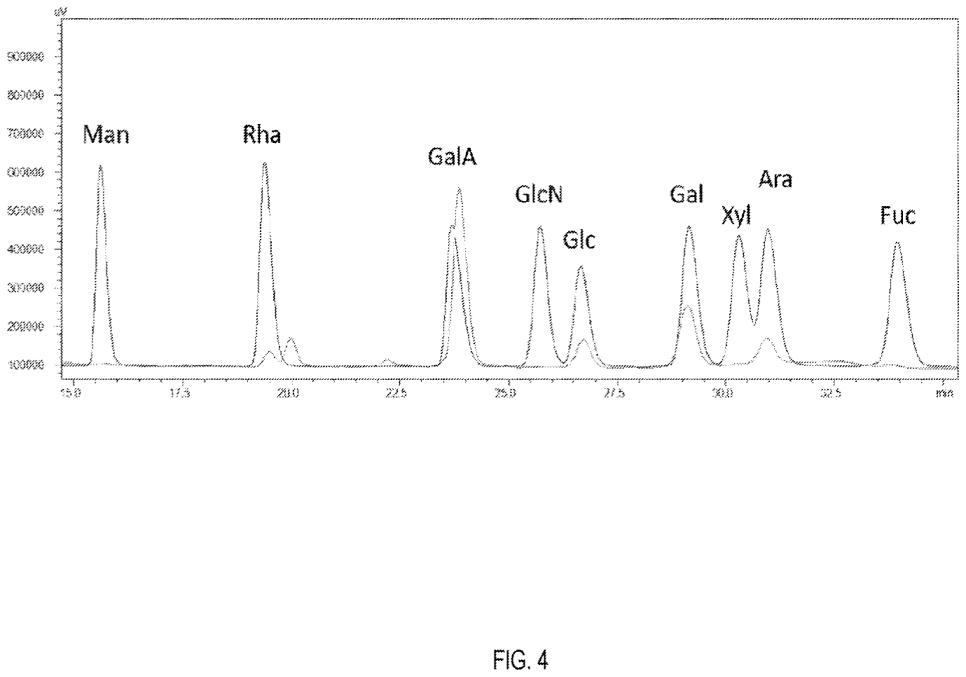

Referring now to FIG. 4, the monosaccharide composition of isolated polysaccharide was determined following hydrolysis and pre-column derivatization by RP-HPLC. Nine standard monosaccharides were separated within 50 min on the XDB-C18 column. The monosaccharide species in the polysaccharide were identified by matching their retention times with those of standard monosaccharides. The results showed that the polysaccharide was composed of rhamnose, galacturonic acid, glucose, galactose and arabinose with a molar ratio of about 2.6:40.1:9.8:16.7:6.1. The proportion was calculated using the peak area of each monosaccharide, corrected by corresponding standards (see Table 1). GalA was the most abundant monosaccharide in the polysaccharide, followed by Gal, Glc, Ara and Rha.

TABLE 1
Molar ratio of rhamnose, galacturonic acid, glucose, galactose and
arabinose in the polysaccharide.

| | Rha | GalA | Glc | Gal | Ara |
|---|---|---|---|---|---|
| peak area of standards | 10217717 | 8998508 | 5951305 | 8928732 | 9165314 |
| peak area of polysaccharide | 268709 | 3608830 | 586019 | 1495086 | 560792 |
| Peak area | 0.0262983 | 0.4010476 | 0.098469 | 0.167447 | 0.0611863 |
| (polysaccharide/Standards) | | | | | |
| Molar ratio | 2.6 | 40.1 | 9.8 | 16.7 | 6.1 |

NMR Spectroscopy

The NMR spectra of the polysaccharides were obtained on a Bruker 800 MHz (18.8 T) standard-bore NMR spectrometer equipped with a ¹H/²H/¹³C/¹⁵N cryoprobe with z-axis gradients. A sample (10 mg) was dissolved in 400 μL of 99.6% D₂O and lyophilized, then repeated twice. ¹H spectroscopy, ¹³C spectroscopy, ¹H-¹H correlated spectroscopy (COSY), ¹H-¹H total correlation spectroscopy (TOCSY), ¹H-¹³C heteronuclear single quantum coherence spectroscopy (HSQC), and ¹H-¹³C heteronuclear multiple bond correlation spectroscopy (HMBC) experiments were all carried out at 298 K.

NMR Spectroscopy Analysis

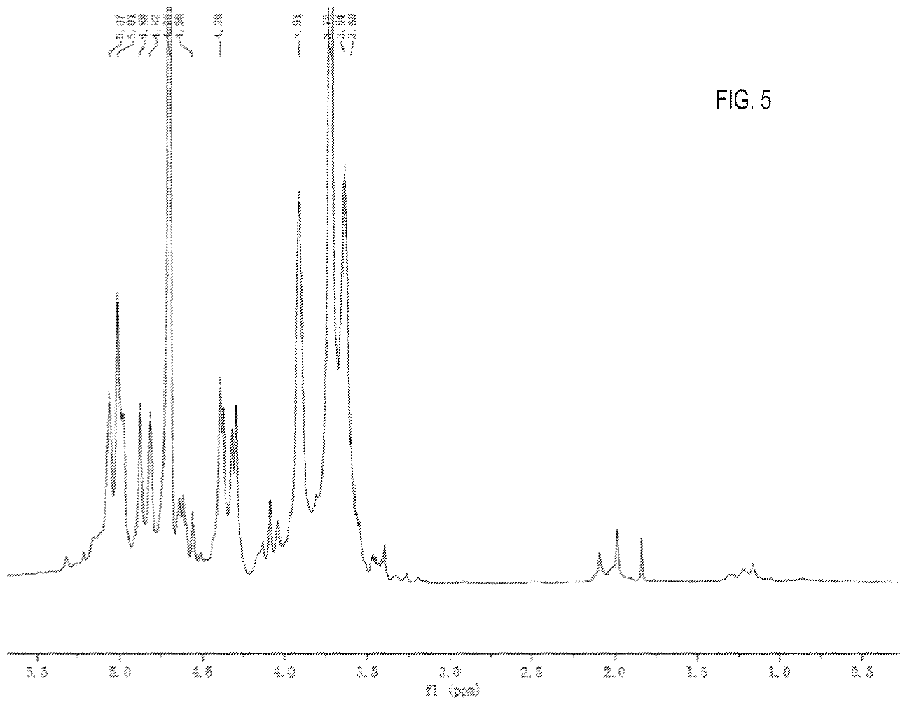

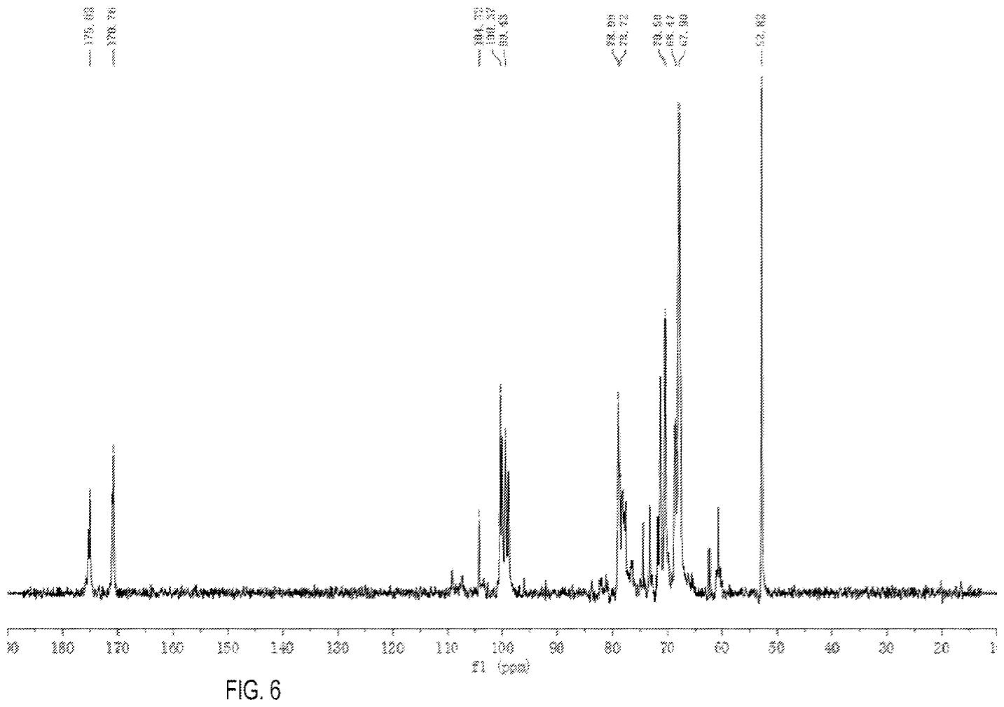

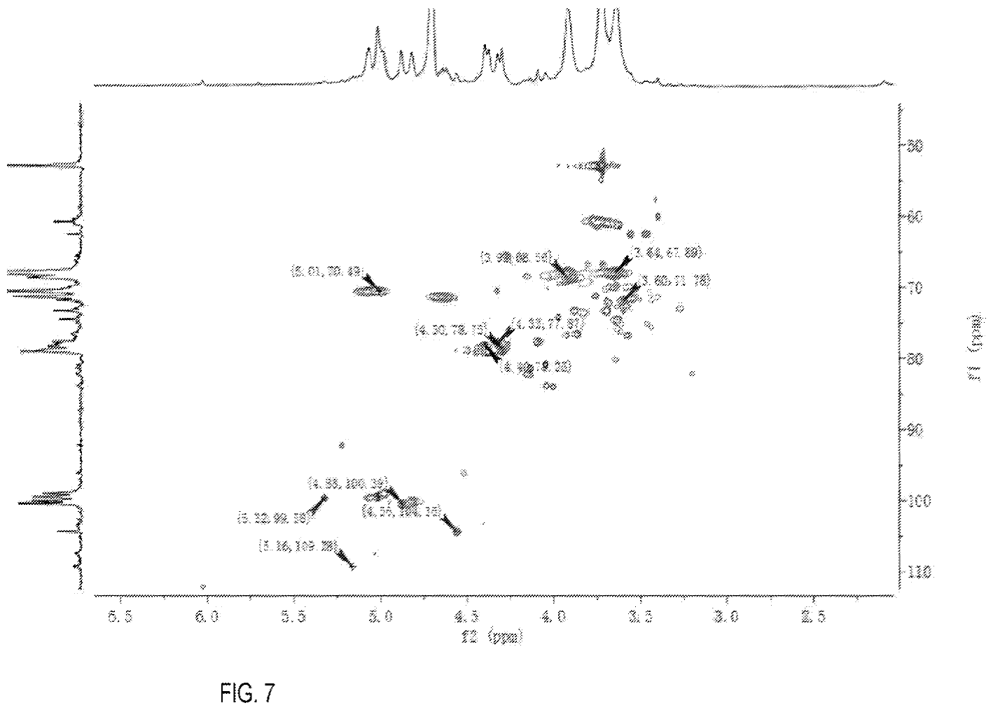

Referring now to FIG. 5, chemical shifts in the ¹H NMR spectrum between δ 4.50 ppm and 5.30 ppm were recognized as anomeric protons region (4.50-5.20 ppm). Other proton peaks were found in the region of 3.30-4.30 ppm. Referring now to FIG. 6, in the ¹³C NMR spectrum, the polysaccharide gave anomeric carbon signals from 97.50 ppm to 108.00 ppm and non-anomeric carbon signals in a broad region from 50.00 ppm to 84.00 ppm. In the ¹³C NMR of the polysaccharide, the signals present at low field 175.08-175.38 ppm were assigned to the carboxyl carbons of GalpA. The signal at 170.76 ppm and that at 52.80 ppm suggested that partial GalA residue might exist as a methyl ester. In ¹H NMR of the polysaccharide, the chemical shifts at δ 3.72 and ˜1.99 ppm attributed to methoxyl and acetyl groups, respectively, indicated that the polysaccharide contain methyl esterified and O-acetylated homogalacturonans. In addition, referring now to FIG. 7, the correlation signals 2.10/20.45 ppm and 2.00/20.15 ppm in the HSQC spectrum confirmed the existence of O-acetyl groups in the polysaccharide. The two doublets suggested that the polysaccharide contained two kinds of O-acetyl groups at the different positions (O-2 and O-3) of GalpA linked residues. The less prominent peaks at the high field (1.23-1.17 ppm) were assigned to the —CH₃ (C6) of rhamnose, indicating a low content of rhamnose in the polysaccharide. The corresponding peak areas in the 1D and 2D NMR spectrum confirmed the presence of homogalacturonan as well as rhamnogalacturonan.

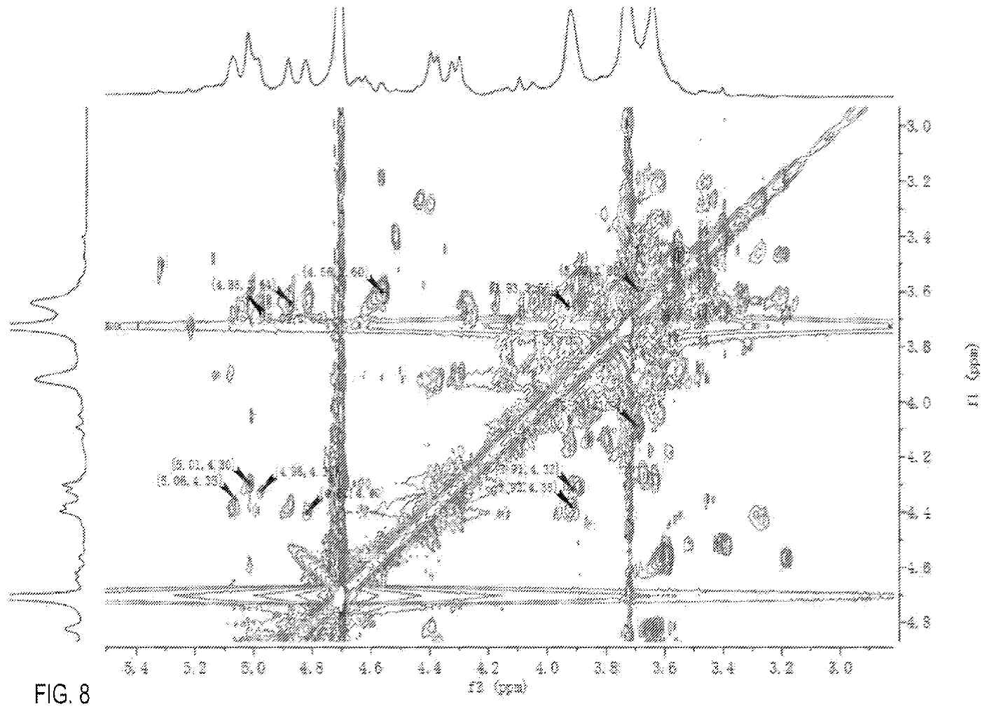

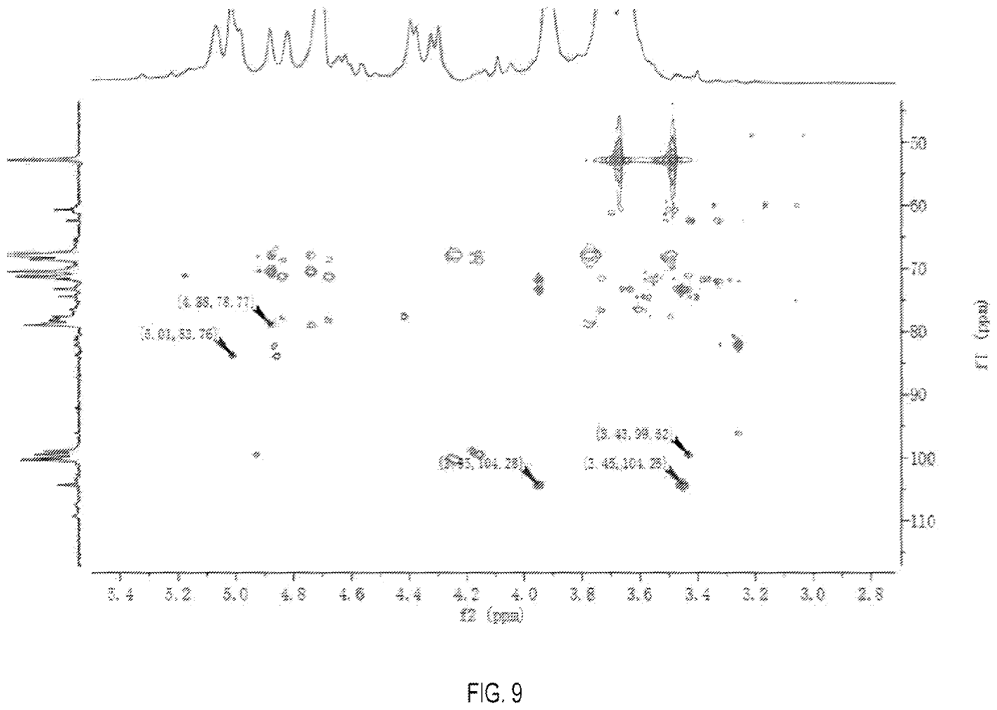

A complete assignment of the signals of non-esterified and esterified carboxyl groups of 1,4-D-GalpA residues is summarized in Table 2. The signals at δ 4.88/100.38 and 5.01/99.43 ppm were assigned to non-methyl and methyl esterified H-1/C-1 of (1,4)-linked GalpA, which also indicated that the GalpA residues possessed an α configuration. Referring now to FIG. 8, the chemical shift of H-2 (3.64 ppm and 3.63 ppm, non-methyl and methyl esterified, respectively) was obtained from the ¹H-¹H correlation (COSY) spectrum. In the same way, H-3 and H-4 can readily be assigned. The signals at 4.82 and 5.07 ppm were attributed to non-methyl and methyl esterified H-5 of (1,4)-linked GapA. All the ¹³C chemical shifts were obtained from the HSQC spectrum. Referring now to FIG. 9, the α-1,4-linkage between GalpA in the main chain was confirmed by a cross peak of H-1 and C-4 at 4.88/79.03 ppm in HMBC spectrum. The chemical shifts at 4.32/77.87 and 4.30/78.71 ppm were assigned to H-4/C-4 of acetyl GalpA.

TABLE 2
Assignment of GalpA carbon/hydrogen signals of polysaccharide

| Residue | | atom | 13 C/ppm | 1H/ppm |
|---|---|---|---|---|
| GalpA | Non-methyl esterified | 1 | 100.38 | 4.88 |
| | | 2 | 67.90 | 3.64 |
| | | 3 | 68.47 | 3.92 |
| | | 4 | 78.72 | 4.39 |
| | | 5 | 70.50 | 4.82 |
| | | 6 | 175.03 | — |
| | Esterified | 1 | 99.43 | 5.01 |
| | | 2 | 70.49 | 3.63 |
| | | 3 | 67.85 | 3.92 |
| | | 4 | 79.03 | 4.37 |
| | | 5 | 70.45 | 5.07 |
| | | 6 | 170.77 | — |
| O—CH3 | — | — | 52.82 | 3.72 |
| O—Ac | — | — | — | 1.99/2.10 |

In the proton spectrum, the rhamnose signals appeared as two doublets, centered at 1.16 and 1.22 ppm, respectively, which were assigned to the 1,2-linked and 1,2,4-linked L-rhamnosyl residues. The C-6 signals at 16.63 and 16.91 ppm were found in HSQC. The anomeric H1/C1 were assigned at 5.32/99.60 ppm by HSQC. The distinct C-1 signal (˜109 ppm) in the anomeric field was ascribed to nonreducing terminals and 1,5-linked L-arabinosyl residues. In addition, the H-1 downfield signals of arabinosyl residues at 5.18, 5.17 ppm indicated that they were α-linked residues. The anomeric H1/C1 was confirmed by HSQC. The ¹H anomeric signals at 4.56 and 4.55 ppm indicated that the galactosyl residues were β-linked, which were further corroborated by the C-1 chemical shift at 104.32 ppm.

Galectin-3 Binding Character of Polysaccharide Composition

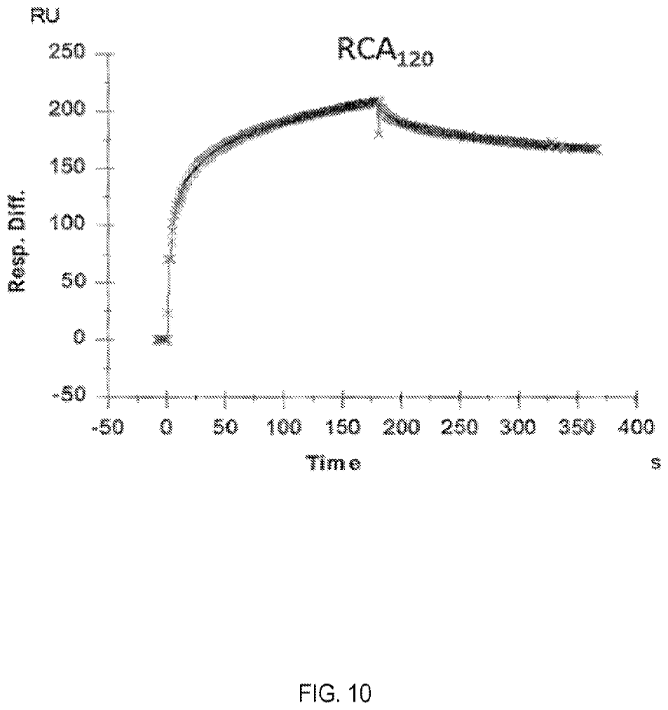

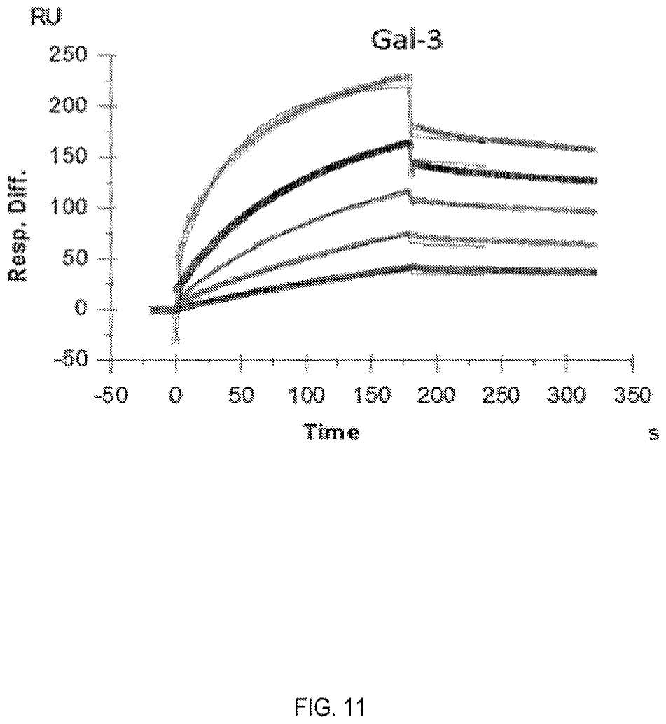

RCA₁₂₀ can be used as a tool to detect β-D-galactose residues. Referring now to FIG. 10, the results of SPR analysis show a smooth binding curve between the polysaccharide composition and RCA₁₂₀, confirming the presence of β-D-galactose in the polysaccharide composition, consistent with the NMR data. The binding kinetics of polysaccharide and galectin-3 interaction were performed by SPR using a sensor chip with immobilized galectin-3 lectin. Sensor grams of galectin-3 binding to different polysaccharide composition dilutions are shown in FIG. 11. Non-specific binding was eliminated by a control flow cell without immobilized galectin-3. The specific binding curves fit well to a 1:1 Langmuir binding model, consistent with a monophasic-binding process. The apparent on (ka) and off (kd) rates for the binding are calculated as 550 (1/ms) and 6.95×10⁻⁴ (1/s), respectively, suggesting quick association and slow dissociation. The binding affinity KD (kd/ka) was calculated to be 1.26 μM, indicating a moderate binding affinity for compound to galectin-3. This gal-3-binding affinity is between that of potato galactan (2.59 μM), and an RG-I domain isolated from ginseng pectin (22.2 nM).

Methods and systems of the present disclosure advantageously provide an agent to inhibit gal-3 binding to cell receptors, blocking its ability to send destructive molecular signals in cancer and other diseases. The pectic polysaccharide in the agent is conveniently isolated from easily and readily available pumpkins, and represents a safe, non-toxic gal-3 inhibitor useful in preventing or reducing galectin-3-mediated diseases and disorders including cardiovascular and renal diseases, cancer, carcinogenesis, fibrosis, and the like.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method of treating a galectin-3 dependent disorder, comprising:
determining that a patient has a galectin-3 dependent disorder; and
administering to the patient a therapeutically effective dose of a therapeutic composition, the therapeutic composition including:
a polysaccharide having a backbone including alternating α-L-rhamnosyl (α-L-Rhap) and α-D-galactopyranosyluronic acid (α-D-GalpA) residues, and a side chain attached to the backbone including β-D-galactan (β-D-Galp), α-L-arabinofuranosyl (α-L-Araf), or combinations thereof.

2. The method according to claim 1, wherein the therapeutically effective dose of the therapeutic composition is administered enterally, parenterally, or combinations thereof.

3. The method according to claim 1, wherein the therapeutically effective dose includes sufficient polysaccharide to inhibit galectin-3 activity, wherein galectin-3 is inhibited at concentrations of the polysaccharide below 2 μM.

4. The method according to claim 1, wherein a β-D-Galp side chain is attached to the backbone at the C-4 carbon of at least one α-L-Rhap of the backbone.

5. The method according to claim 4, where at least one α-L-Araf is attached to the β-D-Galp side chain.

6. The method according to claim 5, wherein the α-L-Araf is attached to the β-D-Galp side chain via the C-3 carbon of the β-D-Galp.

7. The method according to claim 1, wherein the polysaccharide has a structure according to the following Formula I: 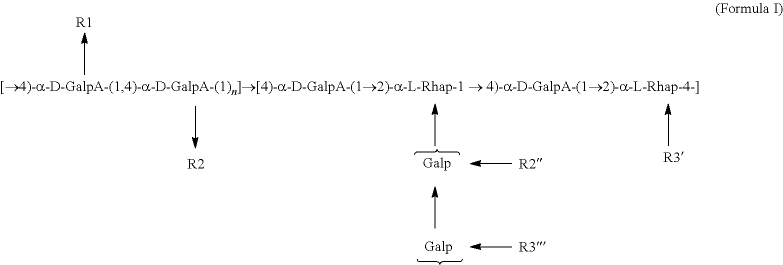
wherein R1 is an H or O-alkyl group, R2 is an H or O-acetyl group, and R3′, R3″, and R3″′ are H, α-L-Araf, or combinations thereof.

8. The method according to claim 1, wherein the polysaccharide is isolated from a member of the genus Cucurbita.

9. The method according to claim 8, wherein the polysaccharide is isolated from C. moschata, C. argyrosperma, C. ficifolia, C. maxima, and C. pepo.

10. The method according to claim 1, wherein the galectin-3 dependent disorder includes galectin-3-mediated diseases and disorders including fibrosis, inflammation, organ damage, impaired organ function, cardiovascular disease, kidney disease, lung disease, cancers, heart disease, elevated blood galectin-3 level, elevated levels of the one or more collagen turnover markers, or combinations thereof.

11. A method of isolating a polysaccharide comprising:
suspending an amount of plant material in an alkali hydroxide solution;
heating the suspension;
isolating a polysaccharide-including supernatant layer from the suspension,
wherein the polysaccharide has a backbone including alternating α-L-rhamnosyl (α-L-Rhap) and α-D-galactopyranosyluronic acid (α-D-GapA) residues, and a side chain attached to the backbone including f-D-galactan (0-D-Galp), α-L-arabinofuranosyl (α-L-Araf), or combinations thereof.

12. The method according to claim 11, wherein a β-D-Galp side chain is attached to the backbone at the C-4 carbon of at least one α-L-Rhap residue of the backbone.

13. The method according to claim 12, further comprising an α-L-Araf attached to the β-D-Galp side chain.

14. The method according to claim 13, wherein the α-L-Araf is attached to the β-D-Galp side chain via the C-3 carbon of the β-D-Galp.

15. The method according to claim 11, wherein the polysaccharide has a structure according to the following Formula I: 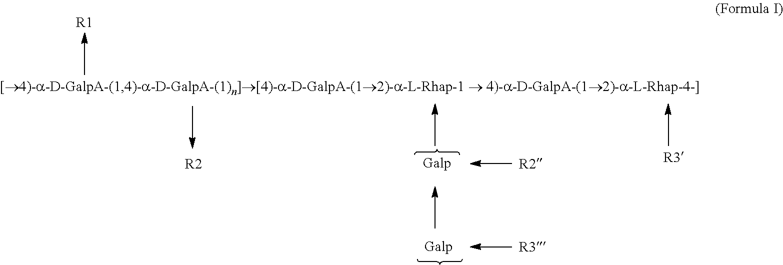
wherein R1 is an H or O-alkyl group, R2 is an H or O-acetyl group, and R3′, R3″, and R3″′ are H, α-L-Araf, or combinations thereof.

16. The method according to claim 11, wherein the plant material is a member of the genus Cucurbita.

17. A therapeutic composition comprising:
a polysaccharide having a backbone including alternating α-L-rhamnosyl (α-L-Rhap) and α-D-galactopyranosyluronic acid (α-D-GapA) residues, and a side chain attached to the backbone including 3-D-galactan (3-D-Galp), α-L-arabinofuranosyl (α-L-Araf, or combinations thereof; and
a pharmaceutically acceptable excipient,
wherein the polysaccharide isolated from a member of the genus Cucurbita.

18. The therapeutic composition according to claim 18, wherein the polysaccharide has a structure according to the following Formula I: 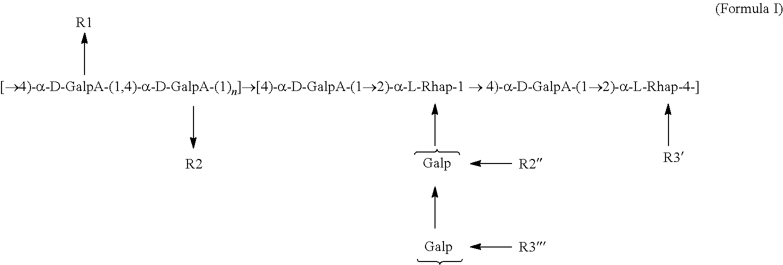
wherein R1 is an H or O-alkyl group, R2 is an H or O-acetyl group, and R3′, R3″, and R3″′ are 1, α-L-Araf; or combinations thereof.

19. The therapeutic composition according to claim 18, wherein the molecular weight of the polysaccharide is about 5 kDa to about 25 kDa.

20. The therapeutic composition according to claim 18, wherein the polysaccharide is produced by a chemical processing method, enzymatic processing method, physical processing method, chemical synthesis, recombinant DNA technology, or combinations thereof.
